(19) United States
(12) Patent Application Publication
(10) Pub. No.: US 2004/0166511 A1
(43) Pub. Date: Aug. 26, 2004

(54) Method for quantifying a ratio between at least two nucleic acid sequences

(76) Inventors: Eveline Catherina Anna Clasina Timmermans, Kaatsheuvel (NL); Bob van Gemen, Almere (NL)

Correspondence Address: TRASK BRITT, P.O. BOX 2550, SALT LAKE CITY, UT, 84110, US

(21) Appl. No.: 10/700,380

(22) Filed: Nov. 3, 2003

Related U.S. Application Data
(60) Provisional application No. 60/425,055, filed on Nov. 8, 2002.

(30) Foreign Application Priority Data
Nov. 8, 2002 (EP) 02079673.6

(51) Int. Cl. C12Q 1/68; C07H 21/04

(52) U.S. Cl. 435/6; 536/24.3

(57) ABSTRACT

The invention provides a method for quantifying an initial ratio of the amounts of at least two nucleic acids of interest in a sample by means of a multiplex nucleic acid amplification reaction, comprising amplifying the nucleic acids of interest in the amplification reaction, measuring the amount of at least two nucleic acids of interest at at least two different time points in the reaction, determining from at least two of the measurements the amplification rate of the at least two nucleic acids of interest, comparing the rates with a reference, and determining from the comparison the initial ratio of the amounts of the at least two nucleic acids of interest in the sample. Preferably, at least one variable factor in the nucleic acid amplification reaction is adjusted in order to allow detectable levels of all nucleic acids of interest to be reached before an amplification and/or detection limit of one or more of the nucleic acids of interest is reached. With the invention, small differences between ratios can be determined, as can direct measurement of the initial nucleic acid ratio. The invention is suitable for determining functioning of a cellular organism, the staging of a disease, and therapeutic activity and/or possible side effects of a compound.

### CROSS-REFERENCE TO RELATED APPLICATION

This application claims the benefit, under 35 U.S.C. § 119(e), of U.S. Provisional Patent Application Serial No. 60/425,055, filed on Nov. 8, 2002, the contents of the entirety of which is incorporated by this reference.

### TECHNICAL FIELD

The invention relates generally to biotechnology, and more particularly to the field of molecular biology.

### BACKGROUND

Detection of nucleic acid is a valuable tool for many biological and medical applications, such as diagnostics. For instance, the presence of a pathogen in an individual can be demonstrated by the detection of foreign, pathogenic nucleic acid in a sample derived from the individual. With such a method, it can often be estimated whether a pathogen is present and, if so, which pathogen is involved. It is often sufficient to qualitatively determine the presence of a certain (pathogenic) nucleic acid sequence. However, in other applications, the amount of nucleic acid should be estimated. For instance, the amount of mitochondrial DNA in a cell is indicative for the number of mitochondria present in the cell. If an amount of mitochondrial DNA in a cell declines over time, it indicates a decrease of mitochondria and a decrease of metabolic activity of the cell. As another example, the amount of an mRNA sequence in a cell is indicative for the level of transcription of the corresponding gene in the cell. A (sudden) change in such level of transcription is indicative for a changed status of an organism, for instance the onset of a disease.

As we have disclosed in PCT International Publication WO 0246470, (mal)functioning of a cellular organism can be determined by determining a ratio, also called a “relative ratio,” of a first endosymbiont cellular organelle nucleic acid and/or gene product thereof in a sample obtained from the organism in relation to the amount of a second nucleic acid and/or gene product thereof. By a “(relative) ratio” is meant the amount of the first endosymbiont cellular organelle nucleic acid and/or gene product thereof in relation to the amount of the second nucleic acid and/or gene product thereof. The ratio can, for instance, be determined by (among other things) dividing the amount of the first nucleic acid or gene product thereof by the amount of the second nucleic acid or gene product thereof, or vice versa. The amount of one or both compounds can also be divided by, or substracted from, a reference value. By “determining functioning of a cellular organism” is meant herein determining whether the cellular organism is in its natural healthy state, or whether the organism is somehow affected, for instance by a disease and/or a (toxic) compound. The disease and/or (toxic) compound may affect the organism to such extent that clinical symptoms are present. Alternatively, the disease or (toxic) compound may have an influence upon the organism while clinical symptoms are not (yet) manifested.

Endosymbiont cellular organelles are those organelles of a eukaryotic cell that are thought to have been derived from prokaryotic bacteria very early on in the evolution of eukaryotic cells. These bacteria (it is thought) have engaged in a symbiosis with early eukaryotic cells, and, at present, eukaryotic cells comprising these endosymbiont organelles in general cannot live without them. None of the present eukaryotic cells would function properly without mitochondria, and most plant cells would at least considered to be malfunctioning if no proplastids, or organelles derived thereof, such as chloroplasts, etioplasts, amyloplasts, elaioplasts or chromoplasts were present. These organelles in general appear to be at least partially self-replicating bodies which, although under some nuclear control, still possess considerable autonomy.

A “ratio of nucleic acids” can be determined by measuring the amount of nucleic acids present in a sample, usually after at least one processing step, like, for instance, amplification of target nucleic acid. After the amounts have been measured, the ratio can be determined by dividing one amount by another.

Minute amounts of target nucleic acid can be detected and quantified by using enzymatic amplification. Examples of enzymatic amplification techniques include PCR,¹ NASBA,² SDA and TMA. Specific amplification of a target nucleic acid sequence can be achieved by adding primer sequences to a reaction. An amplified region can be detected at the end of an amplification reaction by a probe that is specific for the amplified region. Alternatively, an amplified region can be detected during generation of the amplified nucleic acid in the amplification reaction.³ In the latter protocol, a signal of a label attached to a probe can become detectable after the probe has hybridized to a complementary nucleic acid. Examples of such probes that enable real-time homogenous detection in amplification reactions are TaqMan³ and Molecular Beacon probes.^{4;5}

As used herein, “nucleic acid,” “nucleic acid sequence” and “nucleic acid molecule” are used interchangeably.

Quantification of a target nucleic acid sequence is commonly accomplished by adding a competitor molecule, which is amplified using the same primers and which contains sequences that allow discrimination between competitor and target nucleic acid sequence.^{2;6} The ratio between amplified competitor and target nucleic acid sequence can be used to quantify the target nucleic acid sequence. Detection of competitor or target nucleic acid sequence can, for instance, be achieved at the end of the amplification reaction by a probe that is specific for the amplified region of competitor or target nucleic acid sequence or during generation of the amplified nucleic acid in the amplification reaction. In the latter protocol, a signal of a label attached to a probe can become detectable after the probe has interacted with a complementary target nucleic acid and when the target has exceeded a threshold level; the time or cycle number to positivity. In other methods for quantification, the time to positivity can be used for quantification without addition of a competitor.⁷

Hence, in order to determine a ratio of nucleic acid target sequences, the target sequences are mostly amplified. A more precise result can be obtained using the methods disclosed in PCT International Publication WO 0246470 wherein target nucleic acid sequences are amplified simultaneously in a single amplification reaction, preferably in one reaction vessel or tube. This is called “multiplexing of amplification reactions,” or “a multiplex amplification reaction.” By this approach, double spreading in the result due to variation in multiple independent quantitative amplifications is, at least in part, avoided. Generally, double spreading in the result of independent amplification reactions is obtained due to varieties in conditions in different reaction mixtures. For instance, the temperature of the reaction mixture of nucleic acid 1 may be slightly higher than the temperature of the reaction mixture of nucleic acid 2. This may result in a higher yield of nucleic acid 1 and, hence, in a higher ratio of the amount of nucleic acid 1 versus nucleic acid 2 than would have been measured if the temperature of reaction mixture 1 had been exactly the same as the temperature of reaction mixture 2. Because of the temperature difference in the reaction mixtures, the determined ratio is not exactly the same as the real ratio of the two nucleic acids present in the initial sample. Likewise, minute variations in other conditions like for instance the amount of enzyme added can lead to variations in the determined amounts of nucleic acids 1 and 2. Thus, the measured amounts of nucleic acids 1 and 2 may vary independently from each other. Independent variations in the determined amounts may result in an even larger variation in the calculated ratio of the measured amounts. This is called the “double spreading” in the result. Thus, by “double spreading” is meant herein at least one variation in an obtained result, due to a variety of at least one reaction condition in at least two reaction mixtures. For instance, also the total amount of volume may differ slightly between two reaction mixtures.

WO 02/46470 provides a solution to double spreading of results when two or more nucleic acid target sequences are amplified. The number of variables is greatly reduced in a multiplex amplification reaction and more accurate and robust data can be obtained. The initial ratio of at least two nucleic acids is presently determined by measuring distinguishable signals generated during or after an amplification reaction. In the art, multiplex amplification reactions are mainly used for qualitatively detecting the presence of a nucleic acid in a sample.

It is often desired that very small differences in nucleic acid ratios can be measured. Measuring small differences in nucleic acid ratios is, for instance, desired for diagnostic purposes, because in living organisms the amounts of nucleic acids are tightly regulated and even small alterations can involve huge biological consequences. To obtain a diagnosis of the status of an individual, samples taken from the individual at different time points can for instance be investigated for a ratio of mitochondrial DNA versus nuclear DNA. Small alterations of the amount of mitochondrial DNA should be detectable. If such ratio declines slightly over time, for instance by 20%, it indicates that 20% less mitochondria are present. 20% less mitochondria can already involve very severe biological consequences. As another example, if an individual undergoes treatment against a disease, a 20% decline of the presence of (mitochondrial) DNA and/or mRNA is indicative for severe side effects. Therefore, very small changes in ratio should preferably be measurable. With current methods, it is however not always easy to obtain the required sensitivity of discrimination. Especially if nucleic acid is amplified, which is often necessary to obtain a measurable amount, it is not always easy to measure small differences in initial nucleic acid ratios. This is due to many factors which influence the obtained amount of nucleic acid during amplification. Therefore, preferably significant differences between different nucleic acids are currently measured. Moreover, a ratio of nucleic acids after amplification often does not always precisely reflect an initial ratio of the nucleic acids. This is due to differences in amplification rates of different nucleic acids. For instance, a short stretch of nucleic acid is often amplified faster than a second, longer stretch. Because of such problems, it is not always easy with current methods to quantify small differences between nucleic acid ratios. For example, the results of two assessments of HIV-1 viral load that differ by 0.3 log (factor 2) are currently considered to be equivalent results.

### DISCLOSURE OF THE INVENTION

The present invention provides an improved method for quantifying an initial ratio of the amounts of at least two nucleic acids in a sample. With a method according to the invention, small differences between ratios can be determined. Moreover, the invention allows for direct measurement of the initial ratio during and/or after amplification of the nucleic acids. No internal calibrators are needed during amplification. This allows for rapid automatic screening of large amounts of samples. A method of the invention, involving direct measurement of initial nucleic acid ratios, can be combined with high throughput automated practical handling.

The invention provides a method for quantifying an initial ratio of the amounts of at least two nucleic acids of interest in a sample by means of a multiplex nucleic acid amplification reaction, comprising amplifying the nucleic acids of interest in the amplification reaction; measuring the amount of at least two nucleic acids of interest at at least two different time points in the reaction; determining from at least two of the measurements the amplification rate of the at least two nucleic acids of interest; comparing the rates with a reference; and determining from the comparison the initial ratio of the amounts of the at least two nucleic acids of interest in the sample.

By “an initial ratio of the amounts of at least two nucleic acids in a sample” is meant herein the initial amount of a first nucleic acid in the sample in relation to the initial amount of at least a second nucleic acid in the sample. An initial amount of a nucleic acid in a sample is the amount of the nucleic acid in the sample before the nucleic acid has been significantly amplified. The initial amount is preferably essentially equal to the amount of nucleic acid which is present in the sample when the sample is obtained. As used herein, the terms “a ratio of the amounts of nucleic acids” and “a nucleic acid ratio” are equivalent.

Surprisingly, with a method of the invention small alterations in nucleic acid ratios can be measured. Accurate, reliable measurements of small differences of nucleic acid ratios have now become possible. This is very important in order to detect biologically significant changes at an early time point. A method of the invention would not have been expected to be suitable for accurate quantification of small nucleic acid ratio differences because a method of the invention comprises amplification of nucleic acid. As generally thought in the art, the results of nucleic acid amplification vary to some extent due to many factors, such as the amount of enzyme/nucleotides/nucleic acid present, temperature, incubation time, salt concentration, (tissue) origin of a sample, etc. Therefore methods including nucleic acid amplification were not always thought to be suitable for quantification of small differences of nucleic acid ratios. The attempts made in the art to multiplex two or more quantitative amplification reactions into one reaction vessel or tube have therefore often been limited to co-amplify independent quantitative amplifications into one reaction vessel or tube, each quantitative amplification with its own internal calibrator. This approach has been applied to both PCR¹³ and NASBA¹⁴ amplifications. Such methods are, however, not a direct measurement of an initial ratio between two or more independent nucleic acid target sequences but instead are independent quantitative co-amplifications that may have different efficiencies and warrant extremely complex application of internal calibrators for each nucleic acid target sequence. Therefore, it was not always easy to measure small differences of nucleic acid ratios. However, a method of the present invention provides accurate results for small differences in nucleic acid ratios. Amplification rates are now directly connected to an initial ratio of amounts of nucleic acid.

An initial ratio of nucleic acids can also be determined if nucleic acids are present in largely differing amounts. With a method of the invention an initial ratio of the amounts of nucleic acids can be measured within a dynamic range of 3-4 orders of magnitude. This means that even if the amount of one nucleic acid exceeds the amount of another nucleic acid with 3-4 orders of magnitude, the ratio between them can be determined. This is for instance important if a ratio between organelle, or pathogen, nucleic acid is determined in relation to abundant nuclear nucleic acid. If it were not possible to measure a ratio of strongly differing amounts of nucleic acids, small amounts of (important) nucleic acid would sometimes not be detected.

According to the invention, the rates of amplification of nucleic acids are indicative for the original ratio of the amounts of the nucleic acids. This is also the case if a first nucleic acid initially present in a lower amount is amplified faster than a second nucleic acid. This can occur due to many different factors, such as the length of the nucleic acids. After amplification, a higher amount of the first nucleic acid may be present, although the original amount was lower. This sometimes used to be a drawback for quantification by means of amplification, especially by means of multiplex amplification, in the art. However, with a method of the present invention this is no problem, since amplification rates are measured, not the generated amount of amplified nucleic acid.

An amplification rate of a nucleic acid can be determined after a detection level of the nucleic acid has been reached. By a “detection level of a nucleic acid” is meant the minimal amount of the nucleic acid which is required for detection of the nucleic acid with a particular detection method. It will be clear that a detection level is dependent on the detection method used.

Furthermore, an amplification rate of a nucleic acid is preferably determined before an amplification limit and/or detection limit of the nucleic acid is reached. By an “amplification limit of a nucleic acid” is meant the maximal amount of the nucleic acid which can be generated during an amplification reaction. Factors which limit the generation of nucleic acid comprise the amount of nucleotides present. If no more nucleotides are present, no nucleic acid can be generated anymore. By a “detection limit” is meant the maximal amount of generated nucleic acid which can be detected. For instance, if generated nucleic acid is detected with a probe capable of hybridizing to such nucleic acid, the amount of probe can limit the amount of nucleic acid that can be detected. If no more probe is available, the amplification reaction may continue but newly generated nucleic acid cannot be detected anymore.

An amplification rate of a nucleic acid can be determined by measuring the amount of the nucleic acid at two time points after detection level has been reached and before an amplification limit and/or detection limit has been reached, and determining the slope between the two obtained values. Preferably, however, the amount of nucleic acid is measured at least three time points. The slope between the at least two plotted values can be determined by suitable methods known in the art. For instance, a graph can be generated with at least two values, with the X-axis representing time and the Y-axis representing the amount of nucleic acid detected. Subsequently, the slope of a curve essentially connecting the values can be determined. Of course, with current computational techniques, the graph does not have to be physically generated. A slope between a certain set of values can be computed directly, for instance using a computer. In the art, many alternative methods for determining a slope between a certain set of values are known. Such methods can be used in a method of the present invention.

Usually, in an amplification reaction, nucleic acid is firstly linearly amplified where after nucleic acid tends to be non-linearly amplified just before an amplification limit is reached. The same holds true for detection which also increases non-linearly just prior to reaching the detection limit. The amplification rate is preferably determined during linear increase in amplification and detection of a nucleic acid. In a preferred embodiment a method of the invention is therefore provided wherein the time points are chosen within a time interval wherein the amount of detected nucleic acid is linearly increased in time. By linearly increased is meant that the amount of nucleic acid is essentially equally increased within time intervals of the same length.

In terms of the invention, an amplification rate of a nucleic acid means the amount of nucleic acid generated during a certain time interval. The amplification rate is for instance expressed as the increase of nucleic acid per second.

Once the amplification rates of at least two nucleic acids of interest are determined, the rates are compared with a reference. The reference can be generated by performing a method of the invention with different, known, amounts of nucleic acids. Of course, the same kind of nucleic acids as the kind of nucleic acids which are to be quantified by a method of the invention, are preferably used for generation of a reference. Preferably a reference curve is generated, for instance as shown in the examples. Reference curves are preferably generated using multiplex amplification reactions comprising the nucleic acids at varying initial ratios. More preferably, the varying initial ratios comprise essentially the same initial ratio which is expected to be measured in a sample. According to one embodiment, a reference curve is made using nucleic acids from the same kind of cell(s) as the cells wherein the nucleic acids which are to be quantified are present. Preferably, the nucleic acids are of the same kind as the kind of nucleic acids which are to be quantified.

After a reference has been generated, a sample can be submitted to a method of the invention. The amplification rates of nucleic acids of interest are compared to the reference. From such comparison, the initial ratio of the amounts of the nucleic acids of interest in the sample can be determined. This can also be performed if a ratio of amplified nucleic acid has not remained the same as the original ratio of the nucleic acids. If a first nucleic acid is amplified faster than a second nucleic acid, this will also be reflected in the reference values of the nucleic acids.

An even more precise result can be obtained if differences in amplification efficiency between different nucleic acid target sequences are taken into account. In particular in real-time monitored multiplex amplification reactions an increase of distinguishable signals is the result of a competition of multiple nucleic acid target sequences for the amplification reagents in a single reaction, and/or a single vessel or tube, where they are amplified. Many factors influence the amplification efficiency of the nucleic acids to a different extent. Factors which have been found to influence amplification efficiency of a nucleic acid are for example: the properties of the nucleic acid sequence (for instance the GC content and the length of the nucleic acid molecule), modifications of the nucleic acid (for instance methylation), and the secondary and tertiary structure of the nucleic acid. Since amplification of each nucleic acid is influenced by such factors to a different extent, the amounts of amplified nucleic acids often do not precisely reflect the initial amounts of the nucleic acids. Therefore, in current methods in the art the amount of each nucleic acid is often deduced from internal standards. After the amount of amplified nucleic acids has been deduced, a ratio between the amounts is determined. However, in a method of the invention, amplification rates are determined instead of the amount of amplified nucleic acid. Internal standards for determining the amount of amplified nucleic acid are therefore not needed in a method of the invention.

If, however, a first nucleic acid is amplified much more efficiently as compared to a second nucleic acid, there is a risk of the first nucleic acid “overgrowing” the other. In such case, there may be a risk of the second nucleic acid not reaching a detectable amount in time, or sometimes even not at all. If the second nucleic acid does not reach a detectable amount at all, the original ratio of the amounts of nucleic acids may not be determined. It is also possible that a second nucleic acid only reaches detection level when an amplification limit or detection limit or the non-linear phase thereof of a first nucleic acid has already been reached.

Significant differences in amplification rate are often observed between DNA and RNA amplifications. There are many reasons for this, such as among other things the huge difference in structure between DNA and RNA and the need of different kind of enzymes. However, also when amplification reactions are performed with DNA only, or RNA only, differences in amplification efficiency often exist. Therefore, in a preferred embodiment a method of the invention is provided wherein at least one variable factor in the nucleic acid amplification reaction is adjusted in order to allow detectable levels of all nucleic acids of interest to be reached before an amplification and/or detection limit or the non-linear phase thereof of one or more of the nucleic acids of interest is reached. In order to “compensate” for a fast-amplifying nucleic acid, preferably a variable factor is adjusted which affects an amplification efficiency of one nucleic acid of interest to a different extent as compared to another nucleic acid of interest. For instance, a variable factor can be adjusted which mainly affects the amplification efficiency of a nucleic acid which is normally slowly amplified. The amplification efficiency is preferably enhanced. It is of course also possible to adjust a variable factor which mainly affects (preferably decreases) the amplification efficiency of a nucleic acid which is normally rapidly amplified.

Many variable factors can be adjusted in order to affect the amplification rate of at least one nucleic acid. For instance, the length of the amplicons can be adjusted. Preferably, however, at least one primer and/or probe is adjusted. For instance, a primer/probe sequence can be adjusted in order to allow a nucleic acid to be more efficiently amplified/detected. The length of a primer or probe can also be varied. In one embodiment of the invention the variable factor comprises the concentration of at least one primer. Surprisingly, it has been found by the present inventors that the concentration of primer influences nucleic acid amplification rates to a considerable extent, although nucleic acid amplification reactions mostly contain an excess of primer. Apparently, variations in the concentration of primers, even if primers are present in excess, have a considerable effect. Preferably, the concentration of at least one primer is significantly different from the concentration of at least one other primer. With significantly different is meant that the difference between the concentrations is greater than differences due to normal spreading, for instance due to (manual) handling of reagents. Preferably, the concentrations are different to such extent that an amplification efficiency of one nucleic acid of interest is affected to a different extent as compared to another nucleic acid of interest. Preferably the concentration of at least one set of primers capable of annealing to a nucleic acid of interest is adjusted. By a set of primers is meant a set comprising a forward primer capable of annealing to a certain nucleic acid, and a backward primer capable of annealing to the complementary strand of the same (kind of) nucleic acid. More preferably, the concentration of at least one set of primers capable of annealing to a nucleic acid of interest is significantly different from the concentration of at least one other set of primers capable of annealing to a nucleic acid of interest. In the examples is shown that nucleic acid amplification efficiency is strongly influenced when different amounts of primer sets are used. The amount of primer sets can for instance be adjusted such that at least one nucleic acid is optimally amplified.

In another embodiment of the invention, the variable factor comprises the concentration of salt. Surprisingly, it has been found by the present inventors that varieties in salt concentration affect the amplification efficiencies of different nucleic acids in different ways. Hence, by varying a salt concentration, the amplification rate of a specific nucleic acid of interest can be influenced.

With a method of the invention it is possible to influence an amplification rate of a nucleic acid which is normally slowly amplified to such extent that the nucleic acid reaches a detectable level before other nucleic acids have reached their amplification and/or detection limit. The amplification rate of such nucleic acid which is normally slowly amplified can be enhanced. It is also possible to decrease an amplification efficiency of at least one nucleic acid that is normally rapidly amplified.

### BRIEF DESCRIPTION OF THE DRAWINGS

FIG. 1. Fluorescence in time of ROX (chromosomal DNA, grey lines) and FAM (mitochondrial DNA, black lines) fluorescent signal using different ratios of mitochondrial DNA to chromosomal DNA as input. In the lower panel the linear relation between the ratio of signal and the ratio of DNA's is shown.

FIG. 2. Fluorescence in time of FAM (chromosomal DNA, grey lines) and ROX (mitochondrial RNA, black lines) fluorescent signal using different concentrations of primers. Left two panels were performed with 0.4 μM mitochondrial RNA primers and 0.05 μM chromosomal DNA primers. The right two panels were performed with 0.2 μM mitochondrial RNA primers and 0.2 μM chromosomal DNA primers.

FIG. 3. Fluorescence in time of FAM (chromosomal DNA, grey lines) and ROX (mitochondrial RNA, black lines) fluorescent signal using different concentrations of primers. Top two panels were performed with 0.4 μM mitochondrial RNA primers and 0.2 μM chromosomal DNA primers. The bottom two panels were performed with 0.3 μM mitochondrial RNA primers and 0.2 μM chromosomal DNA primers.

FIG. 4. Fluorescence in time of FAM (chromosomal DNA, black lines) and ROX (TIE-1 MRNA, gray lines) fluorescent signal using different concentrations of primers. Left panels were performed with 0.4 μM TIE-1 mRNA primers and 0.1 μM chromosomal DNA primers. The right panels were performed with 0.6 μM TIE-1 mRNA primers and 0.1 μM chromosomal DNA primers. The amount of TIE-1 in vitro generated RNA and plasmid containing the chromosomal DNA target are given in each panel at the bottom of that panel. NT means no template.

FIG. 5. Fluorescence in time of FAM (chromosomal DNA, black lines) and ROX (Siglec-1 mRNA, gray lines) fluorescent signal using different concentrations of primers. Left panels were performed with 0.6 μM Siglec-1 mRNA primers and 0.1 μM chromosomal DNA primers. The right panels were performed with 0.4 μM Siglec-1 mRNA primers and 0.1 μM chromosomal DNA primers. The amount of Siglec-1 in vitro generated RNA and plasmid containing the chromosomal DNA target are given in each panel at the bottom of that panel. NT means no template.

FIG. 6. Two calibration curves of increasing ratios of mitochondrial RNA and chromosomal DNA at 70 mM KCl (left panel) and 90 mM KCl (right panel) in the amplification reaction.

FIG. 7. Ratio of mitochondrial RNA to chromosomal DNA in a healthy individual measured in an amplification with 70 mM KCl (left panel) or 90 mM KCl (right panel) in the amplification reaction.

### DETAILED DESCRIPTION OF THE INVENTION

Preferably, a method of the invention is used for quantifying an initial ratio of the amounts of at least two independent nucleic acid sequences. By independent nucleic acid sequences is meant herein that the sequences are amplified using different primers. No internal calibrators, using the same primers as the nucleic acids of interest, are needed for a method of the invention. In another preferred embodiment, at least one of the nucleic acids of interest comprises RNA. Especially the amount of mRNA is indicative for the rate of transcription of a certain gene and, hence, for (transcriptional) activity of a cell. In yet another preferred embodiment, at least one of the nucleic acids of interest comprises DNA. As shown in the examples, also a ratio of the amounts of a mixture of DNA and RNA can be accurately quantified using a method of the invention.

In one preferred embodiment, a method of the invention is provided wherein the multiplex amplification reaction comprises an amplification reaction wherein nucleic acid is amplified continuously, such as NASBA. An amplification reaction wherein nucleic acid is amplified continuously is preferred, because it has been found that continuously amplifying nucleic acid is particularly suited for measurements of an amplification rate of the nucleic acid. With continuously amplifying nucleic acid, the amount of amplified nucleic acid per time interval is indicative for the amplification rate of the nucleic acid. In amplification reactions which make use of multiple cycles, such as PCR, nucleic acid is not amplified continuously. The time per cycle (i.e. the amount of time for each cycle) has to be chosen carefully. If a nucleic acid of interest is amplified more slowly than the time per cycle, it is not wholly amplified yet when another cycle starts. To avoid the risk of nucleic acid not being wholly amplified during a cycle, the time per cycle necessarily has to be relatively long. In that case, most-or all-nucleic acids of interest will be amplified faster than the time per cycle. Amplified nucleic acid then has to “wait” until a new cycle is started. In a cycle-based amplification reaction the amount of amplified nucleic acid per time interval does therefore not only reflect the rate of amplification of the nucleic acid. It also reflects intervals between cycles wherein no nucleic acid is amplified. Hence, for a method of the invention, a nucleic acid amplification reaction wherein nucleic acid is amplified continuously, such as NASBA, is preferred.

A method of the present invention is suitable for determining (mal)functioning of a cellular organism. As disclosed in our PCT International Publication WO 0246470, (mal)functioning of a cellular organism can be measured by determining the ratio of the amount of a first endosymbiont cellular organelle nucleic acid and/or gene product thereof in a sample obtained from the organism in relation to the amount of a second nucleic acid and/or gene product thereof. The gene product may comprise mRNA. The ratio of the amount of a first endosymbiont cellular organelle nucleic acid and/or gene product thereof in relation to the amount of a second nucleic acid and/or gene product thereof can be accurately determined with a method of the present invention. Therefore, in one embodiment a method of the invention is provided wherein at least one of the nucleic acids of interest comprises an endosymbiont cellular organelle nucleic acid.

(Mal)functioning of a cellular organism can also be measured by determining a ratio of non-endosymbiont nucleic acids. For instance, a ratio between the amount of mRNA and the amount of its corresponding gene is indicative for (transcriptional) activity within a cell. A changing ratio of the amount of a gene and its corresponding mRNA is indicative for an altering status of a cellular organism. The invention thus provides a method for determining functioning of a cellular organism comprising determining the ratio of the amount of a first nucleic acid in relation to the amount of a second nucleic acid in a sample obtained from the organism, wherein the ratio is quantified with a method according to the invention. In one embodiment, the first nucleic acid comprises endosymbiont nucleic acid. Preferably, the ratio of the amount of a first endosymbiont cellular organelle nucleic acid is determined in relation to the amount of nuclear nucleic acid detectable in the sample. Small alterations in the amount of endosymbiont nucleic acid can be detected if a ratio between the endosymbiont nucleic acid and nuclear nucleic acid is determined.

Alterations of a ratio of cellular nucleic acids can be indicative for the onset, or development, of a disease. The staging of a disease can also be determined. For instance, samples from an individual suffering from a disease can be taken regularly. If a disease involves reduction of a certain nucleic acid, the samples can be investigated for the ratio of the amount of the nucleic acid in relation to a (preferably relatively constant) amount of another nucleic acid. If the ratio declines over time, it is indicative for a further development of the disease. If the ratio increases at a later time point, it is indicative for recovery of the patient. Hence, the stage of a disease can be determined. The invention therefore provides a method for determining the staging of a disease, comprising determining the ratio of the amount of a first cellular organelle nucleic acid in a sample obtained from an organism suffering from or at risk of suffering from the disease in relation to the amount of a second nucleic acid, wherein the ratio is quantified with a method of the invention. In one embodiment, the first nucleic acid comprises an endosymbiont cellular organelle nucleic acid, since endosymbiont cellular organelle nucleic acid is often affected when an individual is diseased. The second nucleic acid preferably comprises nuclear nucleic acid, such as for instance a small nuclear ribonucleoprotein nucleic acid, because of its ubiquitous presence. Preferably, the disease involves an HIV-related disease, a tumor-related disease, and/or an angiogenic process. After HIV-infection, an individual often has no clinical symptoms for a significant period. The same is true for the first stage of a growing tumor, or for the first stage from the onset of an angiogenic process. Such angiogenic process is often involved with diseases such as a tumor-related disease, and/or cardiovascular disease. During a first stage of such diseases, the patient is often unaware of it. However, early diagnosis is important for optimal treatment. In such cases, a method of the invention can be used, since a nucleic acid ratio in a cell is often already altered in an early stage of the disease, although no clinical symptoms may be present yet.

A method of the invention for staging of a disease can be used for (early) diagnosis. For instance, people can be routinely tested by a method of the invention within certain time intervals. Alternatively, people can be tested at the moment that they have some clinical symptoms. An alteration in a certain nucleic acid ratio is indicative for a certain degree of disease. The kind of such disease need not be diagnosed by a method of the invention.

In one embodiment, a method of the invention for determining the staging of a disease is provided, wherein the first nucleic acid and the second nucleic acid comprise cellular DNA and RNA. More preferably, the RNA comprises mRNA derived from the DNA. This way, the level of transcription and/or translation can be determined. An alteration of a level of transcription and/or translation, as compared to the natural level of transcription and/or translation, is indicative for an altered functioning of an organism. The altered functioning may be malfunctioning of the organism, because of a disease and/or because of side effects of a certain treatment. The malfunctioning may for instance comprise a decreased level of transcription. Alternatively, the altered functioning may be an improved functioning of the organism, for instance during treatment and/or curing of a disease.

The malfunctioning may as well comprise an increased level of transcription. A disease, or a treatment of a disease, may involve decrement of the amount of cellular DNA. However, the decrement can at least in part be compensated by an increase in transcription of the DNA, at least in the first stage of the disease. This way, the amount of RNA derived from the cellular DNA may not be decreased at all, or relatively less decreased as compared to the amount of the DNA. Symptomatic side effects of the disease or treatment may then not be (fully) sensed yet. However, upon further decrement of the amount of the DNA, the amount of RNA derived from the DNA will eventually also drop significantly. Side effects can then occur. Conventionally, upon manifestation of side effects, a disease is treated or a treatment is reduced or stopped. However, in this conventional way, a patient already suffers from the side effect(s). With a method of the invention, however, side effect(s) involving clinical symptoms can be predicted. For instance, an altered level of transcription and/or translation of cellular nucleic acid are indicative for altered functioning of a cellular organism, for instance malfunctioning of the organism involving (future) side effects. An alteration of a ratio of a first cellular DNA and/or gene product thereof in relation to the amount of a second cellular nucleic acid or gene product thereof is also indicative for altered functioning of a cellular organism.

Other possible uses of the invention lay in candidate drug testing, for beneficial activity and/or side effects of possible medicaments or pharmaceutical compositions such as candidate anti-parasitic compounds, antibiotic compounds, cytostatic compounds, and so on. For example, the invention provides a method for determining therapeutic activity and/or possible side effects of a candidate compound, for example in determining its usefulness for treatment of malfunctioning of a cellular organism, comprising determining the ratio of the amount of a first nucleic acid, preferably an endosymbiont cellular organelle nucleic acid, in a sample obtained from the organism in relation to the amount of a second nucleic acid with a method of the invention. The second nucleic acid preferably comprises a cellular nucleic acid such as a small nuclear ribonucleoprotein nucleic acid, because of its ubiquitous presence. Preferably the organism or an essentially related organism, such as belonging to the same species or genus, has been provided with the compound. If the ratio is altered after the candidate compound is administered to the organism, this indicates therapeutic activity and/or side effects involved with the compound when administered to the organism. Additionally, this also indicates therapeutic activity and/or side effects involved with the compound in an essentially related organism. Therefore, for determining therapeutic activity and/or side effects of a candidate compound for treatment of malfunctioning of a cellular organism, it is not necessary to use exactly the same organism in a method of the invention. An essentially related organism can also be used.

In another aspect, the invention provides a method for determining therapeutic activity and/or possible side effects of a medicament comprising determining the ratio of the amount of a first nucleic acid, preferably an endosymbiont cellular organelle nucleic acid, in a sample obtained from an organism in relation to the amount of a second nucleic acid with a method of the invention. The second nucleic acid preferably comprises a cellular nucleic acid. Preferably the organism has been provided with the medicament.

In terms of the invention, “therapeutic activity” means the capability of at least in part treating a disease. By a side effect of a compound is meant herein another effect than the purpose of the compound. The side effect may be an unwanted effect. For instance, a therapeutic compound may counteract a disease and simultaneously reduce the metabolism of an organism. The reduction of the metabolism is then referred to as a (negative) side effect. Alternatively, a side effect of a compound may be a beneficial effect, like for instance immunity against yet another disease.

In one embodiment of the invention, the therapeutic activity comprises a therapeutic activity against an HIV-related disease and/or a tumor-related disease. The medicament may for instance comprise a cytostaticum, optionally combined with other therapy such as antiretroviral therapy. According to the ATHENA-study in the Netherlands, forty percent of the patients undergoing an antiretroviral therapy need to change antiretroviral therapy because of adverse side effects. Therefore, a method of the invention is very much desired during such therapies, because the method can detect side effects before (severe) clinical symptoms are essentially present. The therapy can then already be stopped and/or changed before the clinical effects are essentially present. In that case the clinical symptoms may not, or to a lesser extent, become present. This will prevent a lot of suffering. Thus, in a preferred aspect a method of the invention is provided wherein the side effects are not essentially manifested at the moment that the method is performed. In terms of the invention, by ‘not essentially manifested’ is meant that a side effect is not (yet), or only partly, manifested by clinical symptoms.

In one aspect a method of the invention is provided wherein the compound or medicament comprises a cytostaticum. Commonly used cytostatica for instance comprise alkylating compounds, antimitotoxic cytostatica, antitumor antibiotica, and topo-isomerase inhibitors. Non-limiting examples thereof comprise chlrambucil, cyclophosphamide, estramustine, ifosamide, melfalanthiotepabusulfan, treosulfancarmustne, lomustinecisplatine, carboplatin, oxaliplatinedacarbazine, vincristine, procarbazine, temozolomide, vinblastine, vindesinedocetaxel, paclitaxeldaunorubicine, doxorubicin, epirubicine, idarubicin, mitoxanthronbleomycin, dactinomycin, mitomycineirinotecan, cladribin, topotecanetoposide, teniposide amsacrine, asparaginase, hydroxycarbamide, pentostatine methotrexate and/or raltitrexed.

During antiretroviral treatment, and/or treatment of tumour-related disease, a nucleoside and/or nucleotide analogue is often used. These analogues involve a high risk of side effects, because they interfere with replication and/or transcription processes in an organism. The amount of endosymbiont cellular organelle nucleic acid is then often altered as well. Therefore, a method of the invention is very suitable when an organism is treated with a medicament involving nucleoside and/or nucleotide analogues.

In one aspect the invention provides a method of the invention wherein the compound or medicament comprises a nucleoside and/or nucleotide analogue. Non-limiting examples of such analogues are fludarabine, mercaptopurine, thioguanine, cytarabine, fluorouracil, and/or gemcytabine. In another aspect a method of the invention is provided wherein the compound or medicament comprises AZT, ddI, ddC, d4T, 3TC, tenofovir and/or abacavir. In a method of the invention, the organism or an essentially related organism has preferably been provided with the compound or medicament.

In yet another embodiment of the invention, the therapeutic activity comprises a therapeutic activity against a process involved in the generation, maintenance and/or breakdown of blood vessels (angiogenic process or angiogenesis). Angiogenesis is an indicator for different aspects. For instance, an increased level of angiogenesis indicates the presence of tumor cells. Tumor cells are dependent on the growth of new blood vessels to maintain expansion of tumor mass. On the one hand, blood vessels are required to carry nutrients to the site of the tumor, whereas on the other hand waste material needs to be transported from the tumor. Therefore, tumors can be indirectly treated with anti-angiogenesis drugs.

In one embodiment a method of the invention for determining therapeutic activity and/or possible side effects of a candidate compound or medicament is provided, wherein the compound or medicament comprises at least one of the following anti-angiogenic drugs: 2ME2, ngiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470, Vitaxin. However, the artisan can think of more drugs that can be used against angiogenesis.

A method of the invention can also be used for (selective) toxin testing. For instance, toxic activity of an herbicide, an insecticide, an anti-parasitic compound and/or an antibiotic compound can be tested by determining whether such compound is capable of altering a ratio of cellular nucleic acids in an organism. The invention thus provides a method for determining toxic activity of a candidate compound for causing malfunctioning of a cellular organism, comprising determining a ratio of the amount of a first nucleic acid in a sample obtained from an organism to an amount of a second nucleic acid with a method of the invention. Preferably the organism or related organism has been provided with the compound. The first nucleic acid preferably comprises an endosymbiont cellular organelle nucleic acid. The second nucleic acid preferably comprises a (ubiquitous) nuclear nucleic acid. With a method of the invention it is for instance possible to test a candidate compound for its capability of causing malfunctioning of a cellular organism, for example, by having a cytostatic or even cytotoxic effect.

In a preferred embodiment, selectivity is also tested, using or applying a method as provided herein (preferably in parallel experiments) on or to a first organism. Preferably a method of the invention is further applied on or to an essentially unrelated second organism. The unrelated second organism preferably belongs to a different family or order. More preferably the unrelated second organism belongs to at least a different class or phylum, most preferably to a different kingdom of organisms. Selectivity aspects can for example be tested by testing a candidate compound in a first target organism, or only in cells of the first organism. The first organism may for instance be a bacterium or parasite. A host or cells thereof is tested as well. Such host is preferably an essentially unrelated second organism, for example a mammal or plant. As another possibility, activity of a candidate compound in a crop plant or cells thereof can be tested by a method of the invention as well as activity of the candidate compound in an essentially unrelated weed plant or cells thereof. This way, a compound can be found with selective toxic or selective therapeutic effects. If a candidate compound appears to be capable of causing malfunctioning of the weed plant, but not causing malfunctioning of the crop plant, the compound may be a potential herbicide.

It is also possible to test normal cells derived from an individual in parallel or comparison with aberrant cells, such as tumour cells derived from the same individual. This way it is possible to detect or screen for a tumour-specific or at least selective cytostatic or cytotoxic compound. Such compound can be used in therapy of the individual or others with similar or related disease.

In one aspect, a method of the invention is provided wherein the first nucleic acid and the second nucleic acid are obtained from a peripheral blood mononuclear cell (PBMC) and/or a fibroblast. Especially the use of PBMCs is preferred because then a blood sample from an organism can be used. A blood sample is easy to obtain and relative large amounts are often available. Therefore, in a preferred embodiment a method of the invention is provided wherein the sample comprises a blood sample.

Furthermore, the invention provides a diagnostic kit comprising at least one means for performing a method according to the invention. Preferably, the kit comprises at least one primer and/or probe set suitable for multiplex amplification and/or detection of nucleic acid related to or derived from endosymbiont cellular organelles and, when so desired, necessary amplification reagents, such as can be found exemplified in the detailed description herein or which are otherwise known in the art. In particular, the invention provides a diagnostic kit wherein the kit comprises more than one primer or probe set for amplification of nucleic acid sequences related to cellular organelles, preferably supplemented with a primer or probe set for the amplification of nucleic acid related to chromosomes, such as a snRNP specific primer or probe. In particular the invention provides a kit comprising at least one primer or probe from Table 1 for multiplex amplification of a nucleic acid sequence related to cellular organelles. In a preferred embodiment, a kit of the invention comprises a significantly different amount of at least one primer as compared to the amount of at least one other primer. Such kit is particularly suitable for performing a method of the invention wherein the concentration of primer is adjusted in order to allow detectable levels of all nucleic acids of interest to be reached before amplification and/or detection limit or the non-linear phase thereof of one or more of the nucleic acids of interest are reached. More preferably, a kit of the invention comprises a significantly different amount of at least one set of primers capable of annealing to a nucleic acid of interest as compared to the amount of at least one other set of primers capable of annealing to a nucleic acid of interest. Preferably, the differences in primer (set) amounts are chosen such that primer concentrations can be used that affect an amplification efficiency of one nucleic acid of interest to a different extent as compared to another nucleic acid of interest. Most preferably, the differences in amount allow for primer (set) concentrations that “compensate” for a fast-amplifying nucleic acid, for instance by enhancing amplification efficiency of a nucleic acid which is normally slowly amplified, or by decreasing an amplification efficiency of a nucleic acid which is normally rapidly amplified.

It is preferred that the amplification reagents, when provided with the kit, comprise an enzyme with reverse transcriptase activity, such as required for PCR or NASBA amplification.

The invention furthermore provides a use of a compound obtainable or selectable by a method according to the invention in the preparation of a medicament, an herbicide, insecticide, anti-parasitic, cytostatic, etc. Preferably, the medicament comprises a medicament against an HIV-related disease, a tumor-related disease, and/or an angiogenic process. A medicament, herbicide, insecticide, anti-parasiticum etc. obtainable or selectable by a method of the invention is also herewith provided.

The following examples are meant to more clarify and illustrate the present invention. They do not limit the scope of the invention in any way. Many alternative embodiments can be performed, which are within the scope of the present application.

### EXAMPLES

### Ingredients and General Methodology

In Table 1, the primers and probes used in the examples are summarized. Standard NASBA nucleic acid amplification reactions were performed in a 20 μl reaction volume and contained: 40 mM Tris-pH 8.5, 70 to 90 mM KCl, 12 mM MgC12, 5 mM dithiotreitol, 1 mM dNTP's (each), 2 mM rNTP's (each), 375 mM sorbitol, 0.105 μg/μl bovine serum albumin, 6.4 units AMV RT, 32 units T7 RNA polymerase, 0.08 units RNAse H, primers and probes in concentrations indicated at the specific examples and input nucleic acid. The amount of KCl in the reactions is between 70 mM and 90 mM depending on the targets that are amplified. The complete mixture, except the enzymes, sorbitol and/or bovine serum albumin was, prior to adding the enzyme mixture, heated to 65° C. for 3 minutes if RNA was amplified and heated to 95° C. for 3 minutes if RNA+DNA or DNA was amplified. After cooling the mixture to 41° C. the enzymes were added. Enzymes are administered in the lid of small (200 μl) Eppendorf tubes and after closing the lid maximally 96 tubes are spun simultaneously for a few seconds to mix the enzyme mix with the other reagents at the bottom of the tubes. After spinning the tubes were placed back at 41° C. and the reagents mixed again by tapping of the tubes. After 3 to 5 minutes at 41° C. the tubes were spun again for a few seconds to collect all fluid at the bottom of the tubes and placed in a fluorimeter. The amplification took place at 41° C. for 90 min in the fluorimeter (Retina^{™} Reader, CytoFluor 4000) and the fluorescent signal was measured every minute (using filter sets specifically for fluorescein and 6-rhodamine).

Cells (fibroblasts and PBMC's) were cultured under standard conditions in standard media known to persons skilled in the art with addition of drugs or putative toxic or stimulating compounds as defined in the examples. Nucleic acids were isolated from the cells with the method described by Boom et al.¹⁶ or with dedicated isolation kits purchased from Qiagen (Qiagen GmbH, Max Volmer Strasse 4, 40724 Hilden, Germany) and used according to the manufacturer's protocols. The isolated nucleic acid was stored at −80° C. until further analysis. Usually the nucleic acid was diluted 10 times with water and of the diluted nucleic acid usually 5 μl was used as input in the NASBA amplification reactions.

### EXAMPLE 1

Different ratios of mitochondrial and chromosomal DNA targets in plasmids were analyzed in this example: 2×10³ Ula DNA/8×10³ Mt DNA, 2×10^{3 Ula DNA/}2×10⁴ DNA, 2×10³ Ula DNA/4×10⁴ Mt DNA, 2×10³ Ula DNA/10⁵ Mt DNA, 2×10³ Ula 2×10⁵ Mt DNA molecules were included. A reaction mix was prepared as described under “used ingredients and general methodology” above with the exception that 64 units T7 RNA polymerase and 9.6 units AMV RT were used and with the addition of 2 units restriction enzyme Msp I. The KCl concentration used in this experiment was 90 mM.

The complete mixture with Msp I, except the amplification enzymes, sorbitol and bovine serum albumin was, prior to adding the enzyme mixture, incubated at 37° C. for 12 minutes to allow Msp I to cut the DNA and subsequently heated to 95° C. for 3 minutes in order to denature the DNA and to allow the primers to anneal. After cooling the mixture to 41° C. the amplification enzyme mixture was added

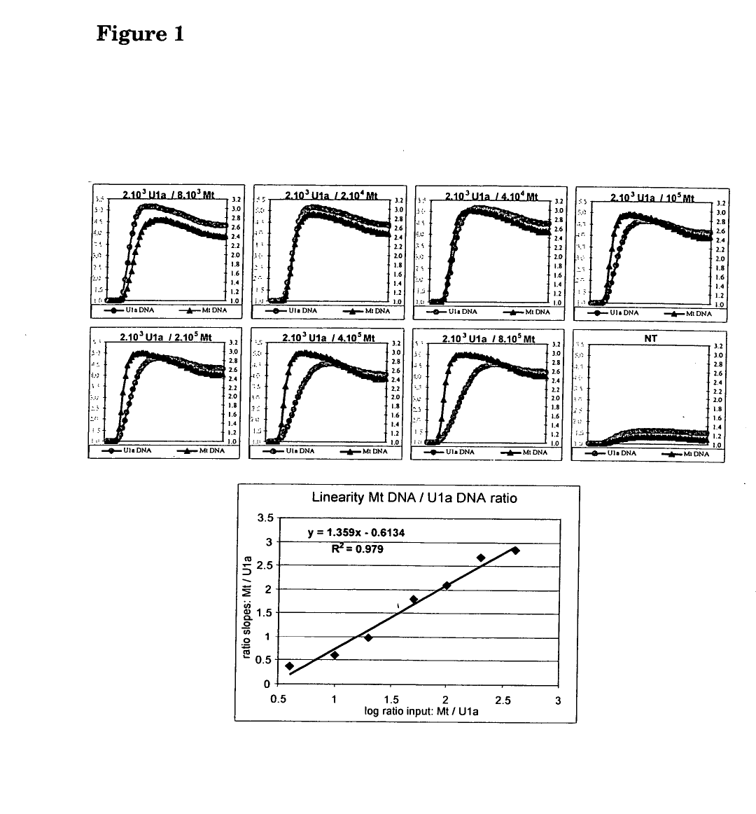

The reaction mix (duplex-mix) contained two sets of primers and beacons: SnrpD p1 and SnrpD p2 (first primer set, each 0.2 μM), and MtD p1 and MtD p2 (second primer set, each 0.3 μM) with beacons SnrpD mb_{—}2 (ROX-labeled) and MtD mb_{—}2 (FAM-labeled) (each 0.05 μM). See table 1 for primer and probe sequences. Filter sets of the fluorimeter (Retina™ reader or CytoFluor 4000 or EasyQ analyzer) were adapted to simultaneously measure the FAM and the ROX-label (485/20 and 530/25 for FAM; 590/20 and 645/40 for ROX). In a duplex reaction with two competing amplifications, the ratio of the slope of the curves of fluorescence in time is proportional to the ratio of the amount of molecules of each amplified species. The results are shown in FIG. 1. The relation between the ratio of the slopes of FAM and ROX signal is linear to the ratio of mitochondrial DNA and chromosomal DNA in the input. This result can be used to generate a calibration curve and the number of mitochondrial DNA copies per cell can be calculated from this standard calibration curve.

### EXAMPLE 2

Two HIV-1 infected patients were treated with a cocktail of anti-viral drugs and their mitochondrial DNA in blood cells was measured as predictor of mitochondrial toxicity. Blood was drawn at week 0, 4, 8, 12, 16, 24, 36 and 48 weeks after the start of therapy. The blood was used to prepare peripheral blood mononuclear cells (PBMC) by Ficoll-Isopaque purification. PBMC were viably frozen in medium plus 5% DMSO and stored in liquid nitrogen or −150° C. until use.

Nucleic acids were extracted from 10⁵ PBMC using the Boom method.¹⁶ Nucleic acids equivalent of 1,000 PBMC were used as input for the one-tube real-time duplex-NASBA that measures both mitochondrial and chromosomal DNA as described in Example 1. The reaction mix (duplex-mix) contained two sets of primers and beacons: SnrpD P1 and SnrpD2 P2 (first primer set, each 0.2 μM), and MtD p1_{—}2 and MtD p2_{—}2 (second primer set, each 0.2 μM) with beacons SnrpD mb_{—}2 (FAM-labeled) and MtD mb_{—}2 (ROX-labeled) (each 0.05 μM). See Table 1 for primer and probe sequences. The result of this assay is expressed as the mitochondrial DNA content per cell (i.e., PBMC) of the patient sample. The results are summarized in Table 2.

The analysis of the duplicate measurements clearly show the reproducibility and accuracy of the duplex test determining the ratio between mitochondrial DNA and chromosomal DNA as a result of optimization of the primer sequences and concentrations in the multiplex amplification reactions. In comparison to Example 1, in Example 2 other primers were used for the amplification allowing a more even use of primer concentration, but at the same time allowing the measurement of rations of more than 400. This is achieved by an imbalance in the efficiency, favoring the chromosomal DNA so that ratio of 400 times more mitochondrial DNA does not compete away the amplification of the chromosomal DNA. The method of the invention avoids the use of internal calibrators and/or separate quantifications of the nucleic acid target sequences and thereby contributes to the design of high throughput tests.

### EXAMPLE 3

In this example, the optimal primer concentrations will be determined for the measurement of the ratio between mitochondrial RNA and chromosomal DNA.

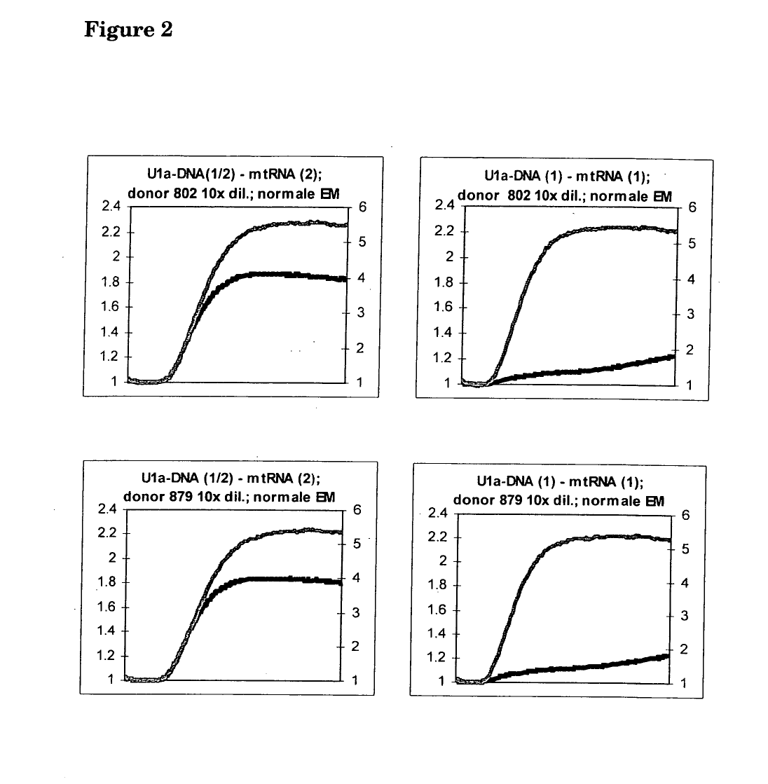

The ratios of mitochondrial RNA target and chromosomal DNA target in two healthy individuals were analyzed in this example. Nucleic acid isolated from PBMC with the Boom method¹⁶ was used as input for the one-tube real-time duplex-NASBA that measures both mitochondrial RNA and chromosomal DNA. The reaction mix (duplex-mix) contained two sets of primers and beacons that were tested at two different concentrations: SnrpD p1 and SnrpD2 p2 (first primer set for chromosomal DNA, and MtR p1_{—}4 and MtR p2_{—}2 (second primer set for mitochondrial RNA) with beacons SnrpD mb_{—}2 (FAM-labeled) and MtR mb (ROX-labeled) (each 0.05 μM). See table 1 for primer and probe sequences. In this experiment primers for amplification of mitochondrial RNA were used at 0.4 μM and 0.2 μM combined with respectively 0.05 μM and 0.2 μM primer concentrations for the chromosomal DNA target sequence. Amplification and detection were performed as in Example 1. Filter sets of the fluorimeter were adapted to simultaneously measure the FAM and the ROX-label (485/20 and 530/25 for FAM; 590/20 and 645/40 for ROX). In a duplex reaction with two competing amplifications, the ratio of the slope of the curves of fluorescence in time is proportional to the ratio of the amount of molecules of each amplified species. The results are shown in FIG. 2.

From the results it is clear that the application of the primer concentrations as used in the left two panels of FIG. 2 render an interpretable result and place the dynamic range of the test right were the expected values for healthy individuals are. The primer concentrations used in the right two panels of FIG. 2 give non-interpretable results because the mitochondrial RNA target sequence is not amplified to detectable levels.

### EXAMPLE 4

In this example, the optimal primer concentrations are determined for the measurement of the ratio between mitochondrial RNA and chromosomal DNA.

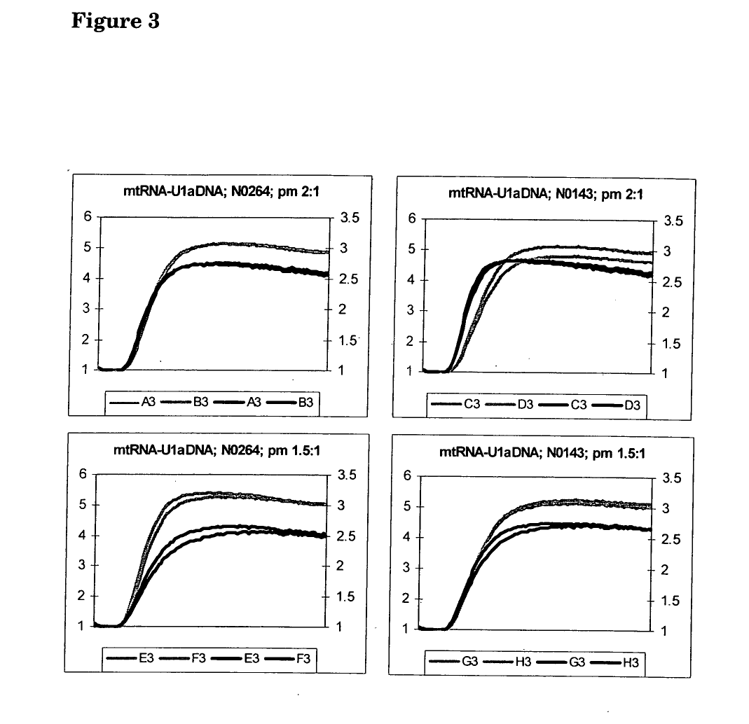

The ratios of mitochondrial RNA target and chromosomal DNA target in two healthy individuals were analyzed in this example. Nucleic acid isolated from PBMC with the Boom method¹⁶ was used as input for the one-tube real-time duplex-NASBA that measures both mitochondrial RNA and chromosomal DNA. The reaction mix (duplex-mix) contained two sets of primers and beacons that were tested at two different concentrations: SnrpD p1 and SnrpD2 p2 (first primer set for chromosomal DNA, and MtR p1_{—}4 and MtR p2_{—}2 (second primer set for mitochondrial RNA) with beacons SnrpD mb_{—}2 (FAM-labeled) and MtR mb (ROX-labeled) (each 0.05 μM). See Table 1 for primer and probe sequences. In this experiment, primers for amplification of mitochondrial RNA were used at 0.4 μM and 0.3 μM combined with 0.2 μM primer concentrations for the chromosomal DNA target sequence. Amplification and detection were done in duplicate and performed as in Example 3. Filter sets of the fluorimeter were adapted to simultaneously measure the FAM and the ROX-label (485/20 and 530/25 for FAM; 590/20 and 645/40 for ROX). In a duplex reaction with two competing amplifications the ratio of the slope of the curves of fluorescence in time is proportional to the ratio of the amount of molecules of each amplified species. The results are shown in FIG. 3.

From the results, it is clear that the application of the primer concentrations as used in the top two panels of FIG. 3 render a clear interpretable result and place the dynamic range of the test right were the expected values for healthy individuals are. The primer concentrations used in the bottom two panels of FIG. 3 give less good results, in particular the bottom left panel were the mitochondrial RNA signal is flattening and becoming close to non-interpretable.

### EXAMPLE 5

Two HIV-1 infected patients were treated with a cocktail of anti-viral drugs and their mitochondrial RNA and DNA in blood cells was measured as predictor of mitochondrial toxicity. Blood was drawn at week 0, 4, 8, 12, 16, 24, 36 and 48 weeks after the start of therapy. The blood was used to prepare peripheral blood mononuclear cells (PBMC) by Ficoll-Isopaque purification. PBMC were viably frozen in medium plus 5% DMSO and stored in liquid nitrogen or −150° C. until use.

Nucleic acids were extracted from 10⁵ PBMC using the Boom method.¹⁶ Nucleic acids equivalent of 1,000 PBMC were used as input for the one-tube real-time duplex-NASBA that measures the ratio between mitochondrial RNA and chromosomal DNA as described in Example 4 and the NASBA that measures the ratio between mitochondrial DNA and chromosomal DNA as described in Example 2. The reaction mix (duplex-mix) for mitochondrial RNA contained two sets of primers and beacons: SnrpD p1 and SnrpD2 p2 (first primer set for chromosomal DNA, and MtR p1_{—}4 and MtR p2_{—}2 (second primer set for mitochondrial RNA) with beacons SnrpD mb_{—}2 (FAM-labeled) and MtR mb (ROX-labeled) (each 0.05 μM). See Table 1 for primer and probe sequences. In this experiment primers for amplification of mitochondrial RNA were used at 0.4 μM combined with 0.2 μM primer concentrations for the chromosomal DNA target sequence. Amplification, and detection were done in duplicate and performed as in Example 3. The result of this assay is expressed as the mitochondrial RNA content per cell (i.e., PBMC) of the patient sample. The results are summarized in Table 3.

The reaction mix for mitochondrial DNA (duplex-mix) contained two sets of primers and beacons: SnrpD P1 and SnrpD2 P2 (first primer set, each 0.2 μM), and MtD p1_{—}2 and MtD p2_{—}2 (second primer set, each 0.2 μM) with beacons SnrpD mb_{—}2 (FAM-labeled) and MtD mb_{—}2 (ROX-labeled) (each 0.05 μM). See Table 1 for primer and probe sequences. Amplification, and detection were done in duplicate and performed as in Example 2. The result of this assay is expressed as the mitochondrial DNA content per cell (i.e., PBMC) of the patient sample. The results are summarized in Table 3.

The analysis of the duplicate measurements clearly show the reproducibility and accuracy of the duplex test determining the ratio between mitochondrial RNA and chromosomal DNA as a result of optimization of the primer sequences and concentrations in the multiplex amplification reactions.

The method of the invention avoids the use of internal calibrators and/or separate quantifications of the nucleic acid target sequences and thereby contributes to the design of high throughput tests.

### EXAMPLE 6

In this example, the optimal primer concentrations are determined for the measurement of the ratio between TIE-1 mRNA and chromosomal DNA.

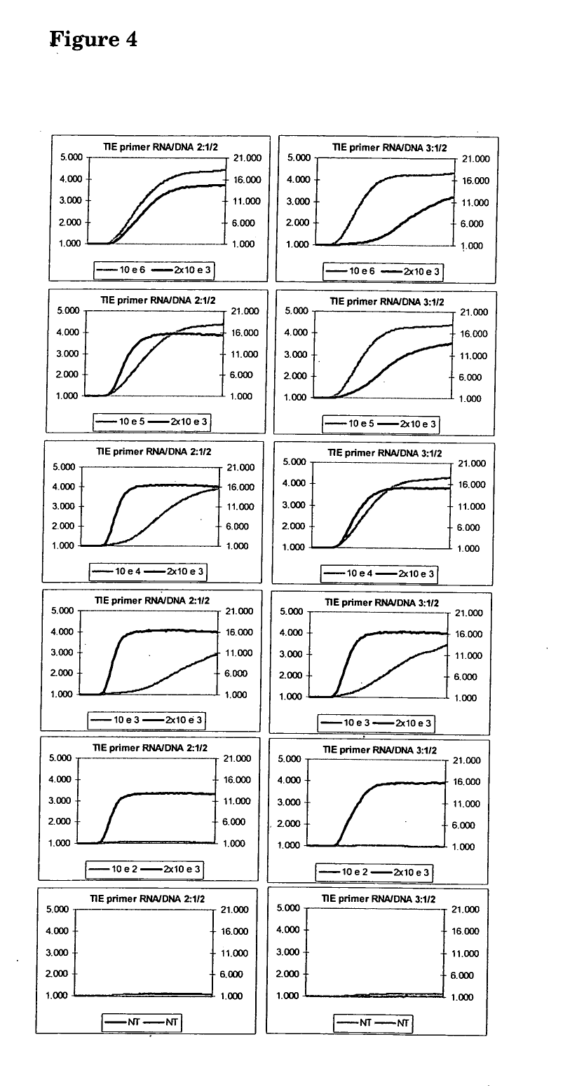

The ratios of TIE-1 mRNA target and chromosomal DNA target in model systems of in vitro generated RNA and plasmids were analyzed in this example. Nucleic acid combinations of in vitro generated part of TIE-1 mRNA and plasmid containing a chromosomal target sequence were used as input for the one-tube real-time duplex-NASBA that measures both TIE-1 mRNA and chromosomal DNA. The reaction mix (duplex-mix) contained two sets of primers and beacons that were tested at two different concentrations: SnrpD p1 and SnrpD2 p2 (first primer set for chromosomal DNA, and TIE p1 and TIE p2 (second primer set for TIE-1 mRNA) with beacons SnrpD mb_{—}2 (FAM-labeled) and TIE mb (ROX-labeled) (each 0.05 μM). See Table 1 for primer and probe sequences. In this experiment, primers for amplification of TIE-1 mRNA were used at 0.4 μM and 0.3 μM combined with 0.2 μM primer concentrations for the chromosomal DNA target sequence. Amplification and detection were done in duplicate and performed as in Example 4. Filter sets of the fluorimeter were adapted to simultaneously measure the FAM and the ROX-label (485/20 and 530/25 for FAM; 590/20 and 645/40 for ROX). In a duplex reaction with two competing amplifications the ratio of the slope of the curves of fluorescence in time is proportional to the ratio of the amount of molecules of each amplified species. The results are shown in FIG. 4.

From the results, it is clear that the application of the primer concentrations as used in the right panels of FIG. 4 render a clear interpretable result and place the dynamic range of the test right were the expected values for healthy individuals are. In particular in the top 3 panels the panels on the right give a much better reflection of the initial input ratio in the ratio of the slopes of the signals of the probes.

### EXAMPLE 7

In this example, the optimal primer concentrations are determined for the measurement of the ratio between Siglec-1 mRNA and chromosomal DNA.

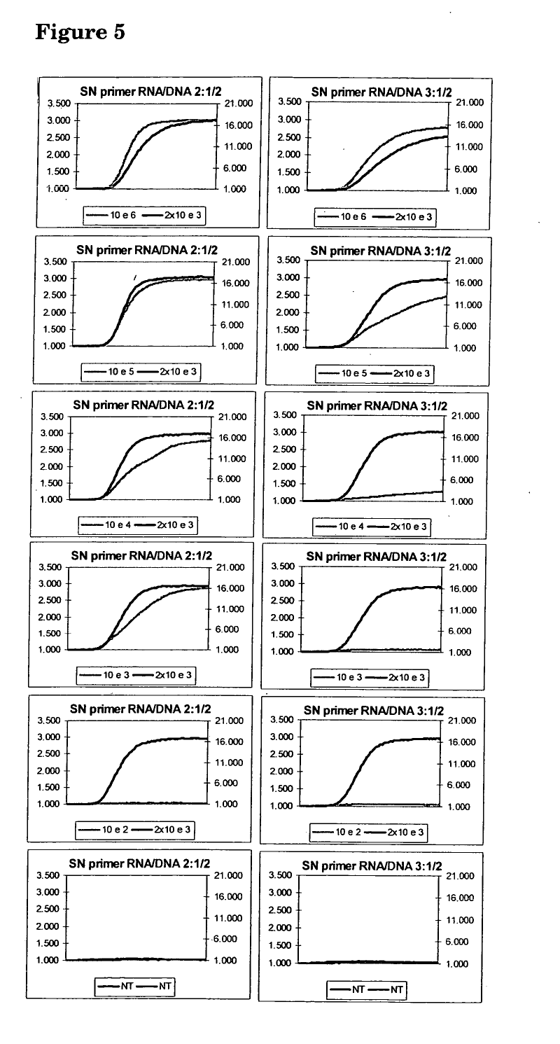

The ratios of Siglec-1 mRNA target and chromosomal DNA target in model systems of in vitro generated RNA and plasmids were analyzed in this example. Nucleic acid combinations of in vitro generated part of Siglec-1 mRNA and plasmid containing a chromosomal target sequence were used as input for the one-tube real-time duplex-NASBA that measures both Siglec-1 mRNA and chromosomal DNA. The reaction mix (duplex-mix) contained two sets of primers and beacons that were tested at two different concentrations: SnrpD p1 and SnrpD2 p2 (first primer set for chromosomal DNA, and Sig p1 and Sig p2 (second primer set for Siglec-1 mRNA) with beacons SnrpD mb_{—}2 (FAM-labeled) and Sig mb (ROX-labeled) (each 0.05 μM). See Table 1 for primer and probe sequences. In this experiment, primers for amplification of Siglec-1 mRNA were used at 0.4 μM and 0.3 μM combined with 0.2 μM primer concentrations for the chromosomal DNA target sequence. Amplification and detection were done in duplicate and performed as in Example 4. Filter sets of the fluorimeter were adapted to simultaneously measure the FAM and the ROX-label (485/20 and 530/25 for FAM; 590/20 and 645/40 for ROX). In a duplex reaction with two competing amplifications, the ratio of the slope of the curves of fluorescence in time is proportional to the ratio of the amount of molecules of each amplified species. The results are shown in FIG. 5.

From the results, it is clear that the application of the primer concentrations as used in the left panels of FIG. 5 render a clear interpretable result and place the dynamic range of the test right were the expected values for healthy individuals are. The results in the left panel are non-interpretable. Other data have shown that the primer concentrations as used in the left panels are the most optimum primer concentrations that render the largest dynamic range and sensitivity.

### EXAMPLE 8

In this example, the optimal salt concentration is determined for a measurement of a ratio between mitochondrial RNA and chromosomal DNA.

At two salt (KCl) concentrations calibration curves of increasing ratios of mitochondrial RNA and chromosomal DNA were made. Also, the ratio of mitochondrial RNA target and chromosomal DNA target in one healthy individual was analyzed with amplifications at different salt concentrations in this example. Nucleic acid isolated from PBMC with the Boom method¹⁶ was used as input for the one-tube real-time duplex-NASBA that measures both mitochondrial RNA and chromosomal DNA. The reaction mix (duplex-mix) contained two sets of primers and beacons that were tested at two different concentrations: SnrpD p1 and SnrpD2 p2 (first primer set for chromosomal DNA, and MtR p1_{—}4 and MtR p2_{—}2 (second primer set for mitochondrial RNA) with beacons SnrpD mb_{—}2 (FAM-labeled) and MtR mb (ROX-labeled) (each 0.05 μM). See Table 1 for primer and probe sequences. In this experiment, primers for amplification of mitochondrial RNA were used at 0.4 μM and 0.2 μM combined with respectively 0.05 μM and 0.2 μM primer concentrations for the chromosomal DNA target sequence. Amplification and detection were performed as in Example 1, with the exception of the KCl concentration, which were, respectively, 70 or 90 mM in the amplification reaction. Filter sets of the fluorimeter were adapted to simultaneously measure the FAM and the ROX-label (485/20 and 530/25 for FAM; 590/20 and 645/40 for ROX). In a duplex reaction with two competing amplifications the ratio of the slope of the curves of fluorescence in time is proportional to the ratio of the amount of molecules of each amplified species.

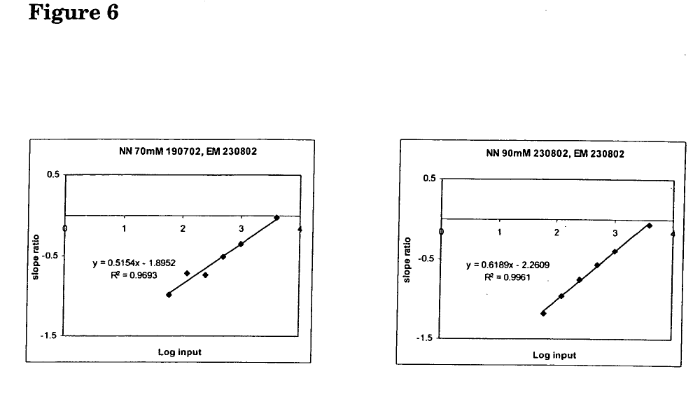

The calibration curves that were made with the different salt concentrations of respectively 70 and 90 mM KCl are shown in FIG. 6. The R², which is a quality measure for the calibration curve differs clearly between the reaction done with 70 mM (R² is 0.9693) and 90 mM (R² is 0.9961) showing that the reactions with 90 mM give a better and more reliable calibration curve.

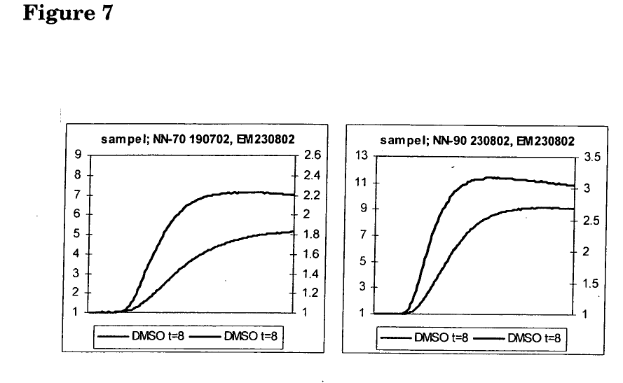

The measurement of the ratio of mitochondrial RNA versus chromosomal DNA in PBMC from a healthy individual at the salt concentrations of 70 an 90 mM in the amplification reaction is shown in FIG. 7. Clearly from the figure it can be concluded that measurement of the ration is more accurate with 90 mM KCl in the amplification reaction (right panel of FIG. 7), as is shown by a steeper increase in signal generation and absolute higher signals (notice the difference in Y-axis scales).

TABLE 1
Sequences of primers and probes used in the examples.

| | |
|---|---|
| Name | Sequence¹ |

| | | |
|---|---|---|
| MtD p1 | 5′ AATTCTAATACGACTCACTATAGGGAGAAGAGCCGTTGAGTTGTGGTA 3′ | (SEQ ID NO:1) |
| | of the enclosed and incorporated hereby sequence listing. | |
| MtD p2 | 5′ TCTCCATCTATTGATGAGGGTCTTA 3′ | (SEQ ID NO:2) |
| MtD p1_2 | 5′ AATTCTAATACGACTCACTATAGGGAAGAACCGGGCTCTGGCATCTTAA 3′ | (SEQ ID NO:3) |
| MtD p2_2 | 5′ GTAATCCAGGTCGGTTTCTA 3′ | (SEQ ID NO:4) |
| MtD mb_2 | 5′ GGACCCCCCACACCCACCCAAGAACAGGGTCC 3′ | (SEQ ID NO:5) |
| SnrpD p1 | 5′ AATTCTAATACGACTCACTATAGGGAGAGGCCCGGCATGTGGTGCATAA 3′ | (SEQ ID NO:6) |
| SnrpD p2 | 5′ TTCCTTACATCTCTCACCCGCTA 3′ | (SEQ ID NO:7) |
| SnrpD2 p2 | 5′ TGCGCCTCTTTCTGGGTGTT 3′ | (SEQ ID NO:8) |
| SnrnpD mb_2 | 5′ CGCATGCTGTAACCACGCACTCTCCTCGCATGCG 3′ | (SEQ ID NO:9) |
| MtR mb | 5′ GCTCCG AAGCTTCTGACTCTTACCTCCC CGGAGC 3′ | (SEQ ID NO:10) |
| MtR p1_4 | 5′ AATTCTAATACGACTCACTATAGGGAGAGGAGACACCTGCTAGGTGTAA 3′ | (SEQ ID NO:11) |
| MtR p2_2 | 5′ GGTGCCCCCGATATGGCGTTCC 3′ | (SEQ ID NO:12) |
| Sig p1 | 5′ AATTCTAATACGACTCACTATAGGGAGAGGACTGGGCCCTCAGCCTGCA 3′ | (SEQ ID NO:13) |
| Sig p2 | 5′ CTGAGGAGACAAGCACCATCA 3′ | (SEQ ID NO:14) |
| Sig mb | 5′ CGTACGAATGACGTGCCCCTGCGAATCGTACG 3′ | (SEQ ID NO:15) |
| TIE p1 | 5′ AATTCTAATACGACTCACTATAGGGAAGAGCTCTCTCCTGTTGGTCCCT 3′ | (SEQ ID NO:16) |
| TIE p2 | 5′ GCATCTCTGTTCATGACTGTGTGA 3′ | (SEQ ID NO:17) |
| TIE mb | 5′ CGTACGCTCAACGCCAGCACGCGCTACCGTACG 3′ | (SEQ ID NO:18) |

1. The T7 promoter part of primer p1 sequences is shown in italics, the stem sequences of the molecular beacon probes are shown in bold. The molecular beacon sequences were labeled at the 3′ end with DABCYL (the quencher) and at the 5′ end with FAM or ROX (the fluorescent labels).

TABLE 2
Ratio between mitochondrial DNA and chromosomal
DNA in PBMC of HIV-1 infected individuals undergoing
anti-viral therapy measured at different time point after
the start of therapy.

duplicate
measurements

| Patient | Week | 1 | 2 | mean value |
|---|---|---|---|---|
| 17 | 0 | 198 | 175 | 187 |
| | 4 | 358 | 391 | 375 |
| | 8 | 347 | 399 | 373 |
| | 12 | 258 | 349 | 304 |
| | 16 | 365 | 348 | 357 |
| | 24 | 351 | 404 | 378 |
| | 36 | 334 | 398 | 366 |
| | 48 | 285 | 378 | 332 |
| 21 | 0 | 325 | 322 | 324 |
| | 4 | 400 | 426 | 413 |
| | 8 | 288 | 283 | 286 |
| | 12 | 330 | 353 | 342 |
| | 16 | 380 | 342 | 361 |
| | 24 | 390 | 439 | 415 |
| | 36 | 462 | 364 | 413 |
| | 48 | 355 | 431 | 393 |

TABLE 3
Ratio between mitochondrial RNA and chromosomal
DNA between mitochondrial DNA and chromosomal
DNA in PBMC of HIV-1 infected individuals undergoing
anti-viral therapy measured at different time point after
the start of therapy.

| duplicate | | |
|---|---|---|
| measurements | Mean | Mean |
| (RNA) | value | value |

| patient | Week | 1 | 2 | RNA | DNA |
|---|---|---|---|---|---|
| A | week 0 | 186 | 229 | 208 | 244 |
| | week 8 | 907 | 938 | 923 | 341 |
| | week 12 | 458 | 367 | 413 | 348 |
| | week 16 | 729 | 839 | 784 | 323 |
| | week 24 | 518 | 498 | 508 | 316 |
| | week 36 | 682 | 632 | 657 | 258 |
| | week 48 | 284 | 292 | 288 | 387 |
| B | week 0 | 125 | 172 | 148 | 209 |
| | week 4 | 274 | 248 | 261 | 77 |
| | week 8 | 576 | 419 | 497 | 71 |
| | week 12 | 439 | 346 | 393 | 68 |
| | week 16 | 129 | 159 | 144 | 69 |
| | week 24 | 201 | 181 | 191 | 47 |
| | week 36 | 453 | 387 | 420 | 109 |
| | week 48 | 402 | 423 | 412 | 191 |

### References

1. Saiki R K, Gelfand D H, Stoffel S, Scharf S J, Higuchi R, Horn G T, Mullis K B, Erlich H A: Primer-directed enzymatic amplification of DNA with a thermostable DNA polymerase. Science 239: 487-491, 1988.

2. Van Gemen B, Van Beuningen R, Nabbe A, Van Strijp D, Jurriaans S, Lens P, Kievits T: A one-tube quantitative HIV-1 RNA NASBA nucleic acid amplification assay using electrochemiluminescent (ECL) labeled probes. J. Virol. Methods 49: 157-167, 1994.

3. Heid C A, Stevens J, Livak K J, Williams P M: Real time quantitative PCR. Genome Res. 6: 986-994, 1996.

4. Tyagi S, Kramer F R: Molecular beacons: probes that fluoresce upon hybridization. Nat. Biotechnol. 14: 303-308, 1996.

5. Leone G, Van Schijndel H, Van Gemen B, Kramer F R, Schoen C D: Molecular beacon probes combined with amplification by NASBA enable homogeneous, real-time detection of RNA. Nucleic Acids Res. 26: 2150-2155, 1998.

6. Piatak M, Luk K C, Williams B, Lifson J D: Quantitative competitive polymerase chain reaction for accurate quantitation of HIV DNA and RNA species. Biotechniques 14: 70-81, 1993.

7. De Baar, M P, Van Dooren, M W, De Rooij, E, Bakker, M, Van Gemen, B, Goudsmit, J, De Ronde, A. Single rapid real-time monitored isothermal RNA amplification assay for quantification of HIV-1 isolates from group M, N, and O. J. Clin. Microbiol. 39(4): 1378-1384, 2001.

8. Richtzenhain L J, Cortez A, Heinemann M B, Soares R M, Sakamoto S M, Vasconcellos S A, Higa Z M, Scarcelli E, Genovez M E. A multiplex PCR for the detection of Brucella spp. and Leptospira spp. DNA from aborted bovine fetuses. Vet. Microbiol. 2002 June 20; 87(2):139-47.

9. Lee J H, Tennessen K, Lilley B G, Unnasch T R. Simultaneous detection of three mosquito-borne encephalitis viruses (eastern equine, La Crosse, and St. Louis) with a single-tube multiplex reverse transcriptase polymerase chain reaction assay. J. Am. Mosq. Control Assoc. 2002 March; 18(1):26-31.

10. Crawford E L, Warner K A, Khuder S A, Zahorchak R J, Willey J C. Multiplex standardized RT-PCR for expression analysis of many genes in small samples. Biochem. Biophys. Res. Commun. 2002 April 26;293(1):509-16.

11. Moutou C, Garders N, Viville S. Multiplex PCR combining deltaF508 mutation and intragenic microsatellite of the CFTR gene for pre-implantation genetic diagnosis (PGD) of cystic fibrosis. Eur. J. Hum. Gen. 2002 April; 10(4):231-8.

12. Jothikumar N, Griffiths M W. Rapid Detection of Escherichia coli 0157:H7 with Multiplex Real-Time PCR Assays. Appl. Environ. Microbiol. 2002 June; 68(6):3169-71.

13. Loitsch S M, Kippenberger S, Dauletbaev N, Wagner T O, Bargon J. Reverse transcription-competitive multiplex PCR improves quantification of mRNA in clinical samples—application to the low abundance CFTR mRNA. Clin. Chem. 1999 May; 45(5):619-24.

14. Deursen P B Van, A W Gunther, C C van Riel, M M van der Eijnden, H L Vos, B van Gemen, D A van Strijp, N M Tackent, and R M Bertina A novel quantitative multiplex NASBA method: application to measuring tissue factor and CD14 mRNA levels in human monocytes Nucl. Acids. Res. 1999 27: e15i-vi.

15. Meijerink J, Mandigers C, van de Locht L, Tonnissen E, Goodthe F, Raemaekers J. A novel method to compensate for different amplification efficiencies between patient DNA samples in quantitative real-time PCR. J. Mol. Diagn. 2001 May; 3(2):55-61.

16. Boom R, Sol C J, Salimans M M, Jansen C L, Wertheim-van Dillen P M, Van der Noordaa J: Rapid and simple method for purification of nucleic acids. J. Clin. Microbiol. 28: 495-503, 1990.

What is claimed is:

1. A method for quantifying an initial ratio of the amounts of at least two nucleic acids of interest in a sample by means of a multiplex nucleic acid amplification reaction, comprising:
amplifying the nucleic acids of interest in the amplification reaction;
measuring the amount of at least two nucleic acids of interest at at least two different time points in the reaction;
determining from at least two of the measurements the amplification rate of the at least two nucleic acids of interest;
comparing the rates with a reference; and
determining, from the comparison, the initial ratio of the amounts of the at least two nucleic acids of interest in the sample.

2. The method according to claim 1, wherein at least one variable factor in the nucleic acid amplification reaction is adjusted in order to allow detectable levels of all nucleic acids of interest to be reached before an amplification and/or detection limit of one or more of the nucleic acids of interest is reached.

3. The method according to claim 2, wherein the variable factor affects an amplification efficiency of one nucleic acid of interest to a different extent as compared to another nucleic acid of interest.

4. The method according to claim 2 or claim 3, wherein the variable factor comprises the concentration of at least one primer.

5. The method according to claim 2, claim 3, or claim 4, wherein the concentration of at least one primer is significantly different from the concentration of at least one other primer.

6. The method according to claim 2, claim 3, claim 4 or claim 5, wherein the variable factor comprises the concentration of at least one set of primers capable of annealing to a nucleic acid of interest.

7. The method according to claim 2, claim 3, claim 4, claim 5, or claim 6, wherein the concentration of at least one set of primers capable of annealing to a nucleic acid of interest is significantly different from the concentration of at least one other set of primers capable of annealing to a nucleic acid of interest.

8. The method according to claim 2, claim 3, claim 4, claim 5, claim 6, or claim 7, wherein the variable factor comprises the concentration of salt.

9. The method according to claim 2, claim 3, claim 4, claim 5, claim 6, claim 7, or claim 8, wherein the nucleic acids of interest comprise independent nucleic acids of interest.

10. The method according claim 2, claim 3, claim 4, claim 5, claim 6, claim 7, claim 8, or claim 9, wherein at least one of the nucleic acids of interest comprises RNA.

11. The method according to claim 2, claim 3, claim 4, claim 5, claim 6, claim 7, claim 8, claim 9, or claim 10, wherein at least one of the nucleic acids of interest comprises DNA.

12. The method according to claim 2, claim 3, claim 4, claim 5, claim 6, claim 7, claim 8, claim 9, claim 10, or claim 11, wherein the multiplex amplification reaction comprises NASBA.

13. A method for determining functioning of a cellular organism, said method comprising:
determining the ratio of the amount of a first nucleic acid in relation to the amount of a second nucleic acid in a sample obtained from the cellular organism,
wherein the ratio is quantified with the method according to claim 1, claim 2, claim 3, claim 4, claim 5, claim 6, claim 7, claim 8, claim 9, claim 10, claim 11, or claim 12.

14. The method according to claim 1, claim 2, claim 3, claim 4, claim 5, claim 6, claim 7, claim 8, claim 9, claim 10, claim 11, claim 12, or claim 13, wherein at least one of the nucleic acids of interest comprises an endosymbiont cellular organelle nucleic acid.

15. The method according to claim 14 wherein the ratio comprises the ratio of the amount of an endosymbiont cellular organelle nucleic acid in relation to the amount of a nuclear nucleic acid in the sample.

16. A method for determining the staging of a disease, said method comprising:
determining the ratio of the amount of a first nucleic acid in a sample obtained from an organism suffering from, or at risk of suffering from, the disease in relation to the amount of a second nucleic acid,
wherein the ratio is quantified with the method according to claim 1, claim 2, claim 3, claim 4, claim 5, claim 6, claim 7, claim 8, claim 9, claim 10, claim 11, or claim 12.

17. The method according to claim 16, wherein the disease is selected from the group consisting of an HIV-related disease, a tumor-related disease, an angiogenic process, and a combination of any thereof.

18. The method according to claim 16 or claim 17, wherein the first cellular organelle nucleic acid and the second nucleic acid comprise DNA and RNA.

19. A method for determining therapeutic activity and/or possible side effects of a compound, said method comprising:
determining the ratio of the amount of a first nucleic acid in a sample obtained from an organism in relation to the amount of a second nucleic acid with the method according to claim 1, claim 2, claim 3, claim 4, claim 5, claim 6, claim 7, claim 8, claim 9, claim 10, claim 11, or claim 12.

20. The method according to claim 19, wherein the therapeutic activity comprises a therapeutic activity against an HIV-related disease, a tumor-related disease, or both an HIV-related disease and a tumor-related disease.

21. The method according to claim 19, wherein the compound comprises a nucleoside and/or nucleotide analogue.

22. The method according to claim 20, wherein the compound comprises a nucleoside and/or nucleotide analogue.

23. The method according to claim 21 or claim 22, wherein the nucleoside and/or nucleotide analogue comprises fludarabine, mercaptopurine, tioguanine, cytarabine, fluorouracil, and/or gemcytabine.

24. The method according to claim 19, claim 20, claim 21, claim 22, or claim 23, wherein the compound comprises AZT, ddI, ddC, d4T, 3TC and/or tenofovir, and/or abacavir.

25. The method according to claim 19, wherein the therapeutic activity comprises a therapeutic activity against an angiogenic process.

26. The method according to claim 25, wherein the compound or medicament comprises at least one of the following drugs: 2ME2, angiostatin, angiozyme, Anti-VEGF RhuMAb, apra (CT-2584), avicine, benefin, BMS275291, carboxyamidotriazole, CC4047, CC5013, CC7085, CDC801, CGP-41251 (PKC 412), CM011, combretastatin A-4 prodrug, EMD 121974, endostatin, flavopiridol, Genistein (GCP), green tea extract, IM-862, ImmTher, interferon alpha, interleukin-12, Iressa (ZD1839), marimastat, metastat (Col-3), Neovastat, octreotide, paclitaxel, penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), tetrathiomolybdate, thalidomide, TNP-470, and Vitaxin.

27. A method for determining toxic activity of a candidate compound for causing malfunctioning of a cellular organism, said method comprising:
determining a ratio of the amount of a first nucleic acid in a sample obtained from an organism in relation to an amount of a second nucleic acid with a method according to claim 1, claim 2, claim 3, claim 4, claim 5, claim 6, claim 7, claim 8, claim 9, claim 10, claim 11, or claim 12.

28. The method according to claim 19, claim 20, claim 21, claim 22, claim 23, claim 24, claim 25, claim 26, or claim 27 wherein the organism or an essentially related organism has been provided with the compound.

29. A method for determining selective activity of a candidate compound against a first organism, said method comprising:
determining therapeutic activity and/or possible side effects of the candidate compound with the method according to claim 19 or claim 20 and/or determining toxic activity with the method according to claim 27.

30. The method according to claim 29 further comprising providing an essentially unrelated second organism with the compound.

31. The method according to claim 30 wherein the first organism comprises a pathogen and the second organisms comprises a host for the pathogen.

32. The method according to any one of claims 13-31, wherein the first nucleic acid comprises an endosymbiont cellular organelle nucleic acid.

33. The method according to any one of claims 13-32, wherein the second nucleic acid comprises a nuclear nucleic acid.

34. The method according to any one of claims 13-33, wherein the first nucleic acid and/or the second nucleic acid is obtained from a peripheral blood mononuclear cell and/or a fibroblast.

35. A diagnostic kit comprising:
at least one means for performing the method according to any one of claims 1-34, and suitable packaging.

36. The diagnostic kit of claim 35, comprising at least one primer or probe selective for amplification and/or detection of a nucleic acid related to or derived from endosymbiont cellular organelles.

37. The diagnostic kit of claim 35 or claim 36, comprising a significantly different amount of at least one primer as compared to the amount of at least one other primer.

38. The diagnostic kit of claim 35, claim 36, or claim 37, comprising a significantly different amount of at least one set of primers capable of annealing to a nucleic acid of interest as compared to the amount of at least one other set of primers capable of annealing to a nucleic acid of interest.

39. The diagnostic kit of claim 36, claim 37, or claim 38, wherein the at least one primer or probe is listed in Table 1.

40. Use of a compound obtainable or selectable by the method according to any one of claims 19-34 in the preparation of a medicament, a herbicide, an insecticide, an anti-parasiticum, an cytostatic agent or cytotoxic agent.

41. A medicament, a herbicide, an insecticide, an anti-parasiticum, an cytostatic agent or cytotoxic agent obtainable or selectable by the method according to any one of claims 19-34.


SEQUENCE LISTING (18 sequences)

SEQ ID NO: 1 - DNA, 48 bp, Artificial sequence
  Synthesized sequence, primer MtD p1
aattctaatacgactcactatagggagaagagccgttgagttgtggta

SEQ ID NO: 2 - DNA, 25 bp, Artificial sequence
  Synthesized sequence, primer MtD p2
tctccatctattgatgagggtctta

SEQ ID NO: 3 - DNA, 49 bp, Artificial sequence
  Synthesized sequence, MtD p1_2
aattctaatacgactcactatagggaagaaccgggctctgccatcttaa

SEQ ID NO: 4 - DNA, 20 bp, Artificial sequence
  Synthesized sequence, primer MtD p2_2
gtaatccaggtcggtttcta

SEQ ID NO: 5 - DNA, 32 bp, Artificial sequence
  Synthesized sequence, primer MtD mb_2
ggaccccccacacccacccaagaacagggtcc

SEQ ID NO: 6 - DNA, 49 bp, Artificial sequence
  Synthesized sequence, primer SnrpD p1
aattctaatacgactcactatagggagaggcccggcatgtggtgcataa

SEQ ID NO: 7 - DNA, 23 bp, Artificial sequence
  Synthesized sequence, primer SnrpD p2
ttccttacatctctcacccgcta

SEQ ID NO: 8 - DNA, 20 bp, Artificial sequence
  Synthesized sequence, primer SnrpD2 p2
tgcgcctctttctgggtgtt

SEQ ID NO: 9 - DNA, 34 bp, Artificial sequence
  Synthesized sequence, primer SnrnpD mb_2
cgcatgctgtaaccacgcactctcctcgcatgcg

SEQ ID NO: 10 - DNA, 34 bp, Artificial sequence
  Synthesized sequence, primer MtR mb
gctccgaagcttctgactcttacctccccggagc

SEQ ID NO: 11 - DNA, 49 bp, Artificial sequence
  Synthesized sequence, primer MtR p1_4
aattctaatacgactcactatagggagaggagacacctgctaggtgtaa

SEQ ID NO: 12 - DNA, 22 bp, Artificial sequence
  Synthesized sequence, primer MtR p2_2
ggtgcccccgatatggcgttcc

SEQ ID NO: 13 - DNA, 49 bp, Artificial sequence
  Synthesized sequence, primer Sig p1
aattctaatacgactcactatagggagaggactgggccctcagcctgca

SEQ ID NO: 14 - DNA, 21 bp, Artificial sequence
  Synthesized sequence, primer Sig p2
ctgaggagacaagcaccatca

SEQ ID NO: 15 - DNA, 32 bp, Artificial sequence
  Synthesized sequence, primer Sig mb
cgtacgaatgacgtgcccctgcgaatcgtacg

SEQ ID NO: 16 - DNA, 49 bp, Artificial sequence
  Synthesized sequence, primer TIE p1
aattctaatacgactcactatagggaagagctctctcctgttggtccct

SEQ ID NO: 17 - DNA, 24 bp, Artificial sequence
  Synthesized sequence, primer TIE p2
gcatctctgttcatgactgtgtga

SEQ ID NO: 18 - DNA, 33 bp, Artificial sequence
  Synthesized sequence, primer TIE mb
cgtacgctcaacgccagcacgcgctaccgtacg